(19) United States
(12) Patent Application Publication
(10) Pub. No.: US 2026/0265830 A1
(43) Pub. Date: Sep. 10, 2026

(54) METHOD OF MEASURING CELL-FREE DNA IN BLOOD OF CANIS FAMILIARIS

(71) Applicant: DeepScan Diagnostics Oy, Helsinki (FI)

(72) Inventors: Katja Kivinen, Helsinki (FI); Juha Kere, Stockholm (SE); Milja Tikkanen, Helsinki (FI); Petra Jaakonsaari, Hyvinkää (FI); Gugan Eswaran, Helsinki (FI); William von der Pahlen, Helsinki (FI)

(73) Assignee: DeepScan Diagnostics Oy, Helsinki (FI)

(21) Appl. No.: 19/069,667

(22) Filed: Mar. 4, 2025

(51) Int. Cl. C12Q 1/6886; C12Q 1/6806; C12Q 1/6809

(52) U.S. Cl. CPC C12Q 1/6886; C12Q 1/6806; C12Q 1/6809

(57) ABSTRACT

A method of measuring a cell-free DNA in blood of Canis familiaris, wherein method includes: separating a plasma from a blood sample of Canis familiaris; spiking the separated plasma with an internal spike; extracting a cell-free DNA from the spiked plasma; measuring a cell-free DNA fragment profile of the cell-free DNA; confirming the internal spike in the cell-free DNA fragment profile; correcting the cell-free DNA fragment profile with the internal spike; monitoring the corrected cell-free DNA fragment profile, wherein the cell-free DNA fragment profile includes set of peaks that range from 100 base pairs up to 700 base pairs; and measuring a cell-free DNA concentration.

### TECHNICAL FIELD

The present disclosure relates to methods of measuring cell-free DNA in blood of Canis familiaris.

### BACKGROUND

The detection and monitoring of various health conditions in animals, particularly in dogs, is an essential aspect of veterinary care. Early and accurate detection of canine health conditions, for example, such as cancer, autoimmune diseases, organ dysfunction, inflammatory disorders, and similar, can significantly improve treatment outcomes and quality of life. While cancer remains a leading cause of mortality among animals, other chronic conditions, such as autoimmune diseases, organ dysfunction, inflammatory disorders, and similar, also significantly impact quality of life for companion animals and cause emotional distress for their owners. Similarly, many chronic conditions are suspected only after changes in a dog's movement and behavior, which again affects the treatment success adversely. Earlier detection of pathological processes is critical to improving treatment outcomes for dogs. Despite advancements in veterinary oncology, many cancers in animals, for example in dogs, are diagnosed only after clinical symptoms arise, which often indicates that disease has progressed to an advanced stage. This delay in diagnosis frequently results in poorer prognoses and limited treatment options, creating an urgent need for effective early detection methods. Reliable cancer screening programs are essential for improving treatment outcomes and enhancing survival rates in canine patients.

However, existing diagnostic techniques in veterinary medicine have not fully addressed these needs.

Conventionally, the existing diagnostic techniques for detecting canine cancer primarily rely on traditional tissue biopsies and imaging techniques, which can be invasive and may not yield timely results. For example, serum biomarker tests have been utilized to monitor cancer progression or treatment response, but their sensitivity and specificity can vary significantly, leading to false negatives or false positives that can complicate diagnosis and management. The limitations of the existing diagnostic techniques highlight a significant gap in the ability to detect cancers early and effectively in dogs.

Conventionally, the existing diagnostic techniques are limited by their reliance on advanced disease symptoms or invasive procedures, which not only delay diagnosis but can also negatively affect patient outcomes.

Existing tests often lack the necessary sensitivity to detect cancers at earlier stages, and their inability to tailor results based on specific breeds or types of cancer diminishes their clinical relevance. Moreover, human oncology is currently undergoing a revolutionary transformation, driven by the rapid expansion of liquid biopsy tests for cancer detection and monitoring. In human medicine, liquid biopsy tests analyzing cell-free DNA (cfDNA) have transformed disease detection and monitoring by enabling non-invasive insight into various health conditions, including cancer, inflammatory diseases, organ health, and similar. However, cfDNA analysis in veterinary medicine remains underutilized, and there are currently no standardized guidelines or reliable tests for comprehensive health screening in dogs. In canine oncology, the lack of reliable tests and standardized screening guidelines means that most patients are only brought to a veterinary clinic after development of clinical signs. This situation emphasizes a pressing need for innovative solutions that can facilitate non-invasive, accurate, and timely cancer detection in dogs, thereby improving the standard of care in veterinary oncology.

Therefore, in light of the foregoing discussion, there exists a need to overcome the aforementioned limitations to enhance early, accurate, and non-invasive detection of diseases in Canis familiaris.

### SUMMARY

The aim of the present disclosure is to provide a method of measuring a cell-free DNA in a blood of Canis familiaris to detect early-stage cancer, monitor its progression, and identify potential precancerous conditions, with breed-specific reference ranges to enhance diagnostic accuracy and sensitivity. The aim of the present disclosure is achieved by a method of measuring a cell-free DNA in a blood of Canis familiaris as defined in the appended independent claim to which reference is made to. The embodiments of the present disclosure substantially enable to improve early detection of canine diseases through cell-free DNA analysis. Additional aspects, advantages, features and objects of the present disclosure would be made apparent from the drawings and the detailed description of the illustrative embodiments constructed in conjunction with the appended claims that follow.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 illustrates steps of a method of measuring a cell-free DNA in a blood of Canis familiaris, in accordance with an embodiment of the present disclosure;

FIG. 2 illustrates an exemplary workflow of processing steps for measuring a cell-free DNA in a blood sample of Canis familiaris, in accordance with an embodiment of the present disclosure;

FIGS. 3A and 3B, illustrate graphical representations of cancer type-specific cell-free DNA fragment size distributions for various canine cancers, in accordance with an embodiment of the present disclosure; and

FIGS. 4A, 4B, 4C, 4D, 4E, and 4F illustrate steps of a graphical representation of breed-specific cell-free DNA concentration levels in healthy controls across different breeds of Canis familiaris, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In an aspect, the present disclosure provides a method of measuring a cell-free DNA in a blood of Canis familiaris, wherein the method comprises:
separating a plasma from a blood sample of Canis familiaris;
spiking the separated plasma with an internal spike;
extracting a cell-free DNA from the spiked plasma;
measuring a cell-free DNA fragment profile of the cell-free DNA;
confirming the internal spike in the cell-free DNA fragment profile;
correcting the cell-free DNA fragment profile with the internal spike;
monitoring the corrected cell-free DNA fragment profile, wherein the cell-free DNA fragment profile comprises a set of peaks that range from 100 base pairs up to 700 base pairs; and measuring a cell-free DNA concentration.

The present disclosure provides the aforementioned aspect of monitoring the cell-free deoxyribonucleic acid (cfDNA) in the blood of Canis familiaris, with specific steps to improve accuracy, reliability, and clinical relevance of the cfDNA analysis. This method addresses key challenges in the cfDNA measurement by incorporating the internal spike for normalization, allowing for precise correction of the cfDNA fragment profile, which is essential for reliable detection and quantification of the cfDNA in a complex biological matrix-like plasma. By separating the plasma from the blood sample and adding the internal spike before the cfDNA extraction, the method enables consistent measurement of the cfDNA fragment profile. Herein, the internal spike acts as a quality control marker, ensuring that any variations or biases introduced during the extraction process or measurement process are minimized. It will be appreciated that confirming the presence of the internal spike in the cfDNA fragment profile allows for correction of potential technical inaccuracies, leading to the corrected cfDNA fragment profile that more accurately represents native cfDNA levels in the blood sample. This correction is particularly advantageous in monitoring the cfDNA, as it accounts for sample processing variability, leading to more reproducible and dependable results across different samples and time points. Moreover, the corrected cfDNA fragment profile provides valuable insights through the set of peaks ranging from 100 base pairs to 700 base pairs, which is relevant for identifying the cfDNA concentration associated with specific biological conditions or diseases. By focusing on this fragment range, the method improves the sensitivity of monitoring the cfDNA, especially in cases where shorter or longer fragments may carry disease-specific information. It will also be appreciated that measuring the cfDNA concentration after correction offers a standardized and quantitative metric for evaluating the cfDNA levels over time. This metric is essential for applications for example, such as early cancer detection, monitoring disease progression, assessing therapeutic response in canine patients, and similar. Other applications include the detection of inflammation, cardiovascular disease, neurodegenerative diseases, autoimmune disorders, infections, and pain.

The term “cell-free DNA” refers to DNA fragments that circulate freely in bloodstream, outside of cells. These fragments originate from cellular breakdown and are released into the bloodstream during normal cell turnover, tissue injury, or disease processes (for example, such as cancer). An analysis of the cfDNA can serve as a valuable biomarker in non-invasive diagnostics, enabling monitoring of conditions for example, such as cancer progression, inflammation, tissue injury, and similar, in the Canis familiaris. Herein, the term “Canis familiaris” is a scientific name for domestic dog, a subspecies of Canis lupus. The Canis familiaris encompasses all breeds of domestic dogs and is widely studied in veterinary medicine and research due to its close relationship with humans, as well as biological and clinical similarities that allow for translational studies relevant to both canine and human health. Herein, the method evaluates efficacy of a liquid biopsy test that uses cfDNA as a biomarker for early cancer detection in the Canis familiaris. Notably, the term “liquid biopsy” refers to a non-invasive diagnostic procedure that involves analysis of biological fluids, for example, such as blood, urine, saliva, cerebrospinal fluid (CSF), and similar, to detect molecular biomarkers, including the cfDNA, that can indicate the presence of disease. In this regard, the liquid biopsy leverages cfDNA fragments, which are released into circulation by cells, including tumor cells, to assess genetic alterations associated with cancer. This approach provides a means for early cancer detection, monitoring of disease progression, and potentially evaluating treatment efficacy without the need for invasive tissue biopsies. The liquid biopsy tests have numerous clinical applications in the context of cancer. These include cancer screening prior to clinical workup, aiding in diagnosis before a definitive diagnosis is made, selecting targeted treatments, detecting minimal residual disease (MRD) following surgery or therapy, monitoring treatment response, and tracking cancer recurrence. Therefore, the liquid biopsy tests have potential to significantly enhance the management of canine cancers by enabling earlier and more accurate detection, as well as more effective monitoring of disease progression and response to therapy. Additionally, this type of analysis allows for more frequent testing and analysis, which is valuable for monitoring the health of the dog. Frequent testing is particularly important as results may sometimes be influenced by various factors, such as activity level of the dog, health status of the dog, and similar, at the time of sample collection. The advantages of measuring cfDNA compared to existing blood panel biomarkers include its wider dynamic range and its rich informativeness. The concentration of cfDNA in blood or another source material can give some information about the type of pathological process behind its increase. cfDNA also carries information about the tissue of origin and the pathological processes that have caused its release into the bloodstream. This information is in the form of genetic and epigenetic changes that can be readily analysed using sequencing and PCR-based technologies.

Throughout the present disclosure, the term “plasma” refers to a liquid component of blood that remains after the removal of blood cells (including red blood cells, white blood cells, and platelets) through centrifugation. Herein, the plasma is separated by centrifuging the blood samples of the Canis familiaris to remove cellular components. The centrifugation process separates the denser blood cells from lighter plasma, allowing the plasma, which contains the cfDNA, to be collected as a supernatant. The resulting plasma is then used as a sample medium for extraction and analysis of the cfDNA. This step establishes a consistent sample type, which is important for reliable measurement and comparison of the cfDNA across different samples. It will be appreciated that this approach minimizes contamination from cellular DNA, as cellular components are effectively removed, thereby enhancing the purity and accuracy of the cfDNA measurement. It will also be appreciated that by standardizing the plasma as sample type, the method achieves reproducibility across various blood samples of the Canis familiaris, enabling consistent levels of the cfDNA to be measured without interference from variable cellular content. Furthermore, by isolating the plasma promptly, the method reduces the risk of degradation of the cfDNA, thereby ensuring that the cfDNA remains intact for analysis and enhancing the reliability of the detection and quantification process.

Optionally, the blood sample is collected from one of the following: vena cephalis, vena saphena lateralis, vena jugularis. In this regard, the method specifies that the blood sample is collected from one of three accessible veins: the vena cephalica, the vena saphena lateralis, or the vena jugularis. Each of these veins is selected for its suitability for blood collection in canine, allowing veterinary professionals to draw blood efficiently while minimizing stress and discomfort for the animals. Typically, the blood sample is obtained using a syringe, ensuring a clean and uncontaminated sample. Once collected, the blood sample is placed in appropriate collection tubes, for example, such as Ethylenediaminetetraacetic acid (EDTA) tubes, serum tubes, heparin tubes, or commercial circulating cell-free (ccf) DNA tubes, which preserve integrity of the blood sample for subsequent plasma separation and extraction of the cfDNA. This approach standardizes a collection process of the blood sample. It will be appreciated that specifying the collection of the blood sample from one of the following veins: the vena cephalica, the vena saphena lateralis, or the vena jugularis, facilitates efficient blood sampling, thereby minimizing stress and discomfort for the canine patient. It will also be appreciated that these veins are chosen for their anatomical accessibility and sufficient blood volume, ensuring that a consistent and adequate sample size is obtained for accurate analysis of the cfDNA. Furthermore, by standardizing the collection site, the method enhances reproducibility and comparability of results across different samples, which is essential for reliable data interpretation in clinical studies.

In an exemplary methodology, blood samples may be collected from a dog, wherein the blood sample comprises a minimum volume of 3 millilitres (mL) of a total volume of the blood sample. The blood may be drawn from the vena cephalica. Moreover, the plasma isolation procedure of the blood sample may utilize a double centrifugation protocol: an initial centrifugation at 1800 g for 10 minutes at room temperature, followed by a second centrifugation at 1800 g for 10 minutes at room temperature. Following the centrifugation process, the separated plasma may be then transferred to clean DNA and may be stored at −20° C. until ready for shipment. This methodology ensures integrity of the cfDNA throughout the collection and storage process, facilitating accurate and reliable downstream analysis. It is important to store plasma frozen and in ship it frozen to maximize the cfDNA to be recovered. Any freeze-thaw cycles along the way decreased cfDNA concentration further.

A technical effect of the aforementioned feature is that it ensures the blood samples have been collected from standardized anatomical sites, which enhances the consistency and reliability of analysis of the cfDNA.

Throughout the present disclosure, the term “internal spike” refers to an intentional addition of a known quantity of a specific substance (often a biomolecule or an analyte) to the separated plasma. Notably, the internal spike serves as a control reference during subsequent extraction and quantification of the cfDNA. The internal spike is added to the separated plasma before the extraction process begins. The internal spike may be, for example, a synthetic cfDNA fragment, a known concentration of a DNA fragment of a specific size and sequence, and similar, which is not naturally present in the separated plasma. This involves selecting a specific quantity of the internal spike DNA, which is then mixed with the plasma sample. The purpose of spiking the plasma with the internal spike is to correct for potential variations in the efficiency of the cfDNA extraction process. These variations can arise from factors for example, such as sample handling, extraction methodology, batch differences, or similar. By including the internal spike of known size and concentration, the method ensures that the measurement of the cfDNA is accurate and reliable, thereby enhancing validity of the study results. It will be appreciated that the use of the internal spike minimizes the impact of technical variability, leading to more reproducible results that are essential for robust data interpretation in clinical applications.

Throughout the present disclosure, the extraction process optionally involves utilizing a commercial extraction kit, for example, such as the Qiagen QiaAmp® circulating cell-free DNA mini kit, which is specifically designed to efficiently isolate the cfDNA from the spiked plasma. The spiked plasma sample is treated with reagents that separate the cfDNA from cellular debris and other components. This process may include binding the cfDNA to a solid phase, washing to remove impurities, and eluting the cfDNA into a suitable buffer. The presence of the internal spike allows for adjustments to be made based on its recovery, thereby validating the extraction process. Extracting the cfDNA from the spiked plasma is necessary to analyse genetic material that circulates in the bloodstream, which can provide valuable insights into the health status of the Canis familiaris and is useful in various diagnostic and research applications. It will be appreciated that the extraction process allows for the accurate quantification of the cfDNA, as the presence of the internal spike serves as a reference point for assessing extraction efficiency.

Optionally, the extracted cell-free DNA is diluted with an elution buffer. In this regard, the term “elution buffer” refers to a specific solution used in molecular biology and biochemistry to facilitate release and purification of nucleic acids, such as DNA, from solid-phase matrices or other biological materials. The elution buffer is formulated to provide optimal conditions for solubilizing nucleic acids, ensuring their stability and integrity during and after the extraction process. Typically, the elution buffer may contain components, for example, such as salts, buffering agents, detergents, stabilizers, and similar, that prevent degradation of the nucleic acids. In an implementation, a suitable elution buffer is selected, which is often provided by the extraction kit (such as the Qiagen QiaAmp® circulating cfDNA mini kit), with a composition optimized for maximum DNA stability and yield. Then, a defined volume of the elution buffer is mixed with the extracted cfDNA. For example, the extracted DNA may be diluted to a volume of 50 microliters (μL) with the elution buffer, ensuring thorough mixing to achieve homogeneity. Following dilution, the quality and concentration of the cfDNA can be assessed using techniques such as spectrophotometry or fluorometry. This step is essential to verify that the dilution has resulted in an appropriate concentration for downstream applications. Herein, the concentration of the cfDNA obtained from the extraction process may vary significantly based on source and conditions of the extraction. Thus, dilution standardizes the concentration of the cfDNA, allowing for uniformity across the blood samples, which is essential for comparative analyses and interpretation of results. A technical effect of using the elution buffer to dilute the extracted cfDNA is that it preserves DNA integrity and stability, thereby enabling accurate quantification and enhancing the reliability of downstream genetic analyses across clinical and research applications.

Throughout the present disclosure, the term “cell-free DNA fragment profile” refers to a detailed characterization of size distribution, quantity, and structural attributes of DNA fragments found in the cfDNA. The cfDNA fragment profile provides insight into the molecular composition of the cfDNA, typically isolated from the plasma or other bodily fluids, by identifying lengths and relative abundances of the DNA fragments within the blood sample. The cfDNA fragment profile is commonly used in diagnostic and research applications, as variations in fragment size and distribution can reflect biological processes, tissue origin, or pathological conditions. Beneficially, measuring the cfDNA fragment profile is important as the size and distribution of DNA fragments can provide valuable insights into their origin, integrity, and potential health implications. For instance, the cfDNA fragment profile may differ in normal versus diseased states, such as in cancer, inflammatory conditions, or autoimmune diseases. These variations can reveal information about the condition, tissue of origin, and other health factors.

For example, distinct cfDNA profiles may be associated with infection, chronic fatigue syndrome, metabolic disorders, and similar providing insights into underlying health conditions and systemic stress responses. Herein, measuring the cfDNA fragment profile, specific analytical tools, for example, such as capillary electrophoresis, high-resolution electrophoresis platforms, and similar, are used. The extracted cfDNA is processed through these instruments to generate the cfDNA fragment profile that indicates the distribution and sizes of DNA fragments within the blood sample. This process involves preparing the blood sample with fluorescent markers and separating the DNA fragments by size, resulting in the cfDNA fragment profile that can be analysed to identify characteristic patterns. A technical effect of measuring the cfDNA fragment profile is that it enables the detection of specific fragment patterns, improving the ability to diagnose, monitor, and study biological conditions based on characteristics of the cfDNA.

Throughout the present disclosure, to confirm the internal spike, the extracted cfDNA fragment profile is analyzed to check for the presence and characteristics of the spike, such as its size and concentration. In this regard, techniques such as gel electrophoresis, spectrophotometry, capillary electrophoresis, or similar, can be used to visualize and confirm the internal spike in the cfDNA fragment profile. Such techniques are well-known in the art. The internal spike should appear within a predictable range in the cfDNA fragment profile, correlating with its known properties (such as fragment length and concentration). Any deviation from expected values can indicate extraction inefficiency or handling errors, prompting corrections or adjustments in data interpretation. It will be appreciated that this step ensures the precision and reproducibility of results across different batches and blood samples of the Canis familiaris, thereby making data more robust for diagnostic and research applications where precise cfDNA levels are essential. A technical effect of confirming the internal spike in the cfDNA fragment profile is that it enhances the reliability and reproducibility of analysis of the cfDNA by providing a control reference for extraction efficiency and handling consistency.

Throughout the present disclosure, to correct the cfDNA fragment profile with the internal spike, the extracted cfDNA is analyzed, and the internal spike is identified within the cfDNA fragment profile based on its known size, concentration, and fragmentation pattern. The measured presence and intensity of the internal spike are compared to the expected values, and adjustments are made to the cfDNA fragment profile accordingly. This can involve scaling or normalizing the entire cfDNA fragment profile or applying corrections to specific fragment sizes, depending on the analysis method employed. For example, discrepancies in the spike's recovery could lead to a recalibration of the overall cfDNA fragment profile, accounting for any extraction inefficiencies. The purpose of this step is to correct for any potential biases or errors that may arise during the extraction process of the cfDNA, for example, such as variations in extraction efficiency, sample handling, batch-to-batch differences, or similar. These factors can introduce discrepancies in the cfDNA fragment profile. By using the internal spike, which has a known quantity, size, and concentration, one can compare its recovery to the expected values, thereby identifying and correcting for these variations. It will be appreciated that using the internal spike to validate and correct the cfDNA profile enhances the accuracy and precision of interpreting cfDNA-based biomarkers, thereby supporting more reliable diagnostic outcomes for the Canis familiaris.

Throughout the present disclosure, the term “corrected cell-free DNA fragment profile” refers to adjusted representation of the size distribution and concentration of the cfDNA fragments in a biological sample after accounting for variations in the extraction process, sample handling, or other experimental factors. The term “set of peaks” refers to a group of intensity maxima observed in a chromatographic data, an electrophoretic data, a spectrometric data, and other analytical profiles, each corresponding to a specific cfDNA fragment or a group of fragments with similar molecular weights, measured in base pairs. Herein, the set of peaks may, for example, lie in a range from 100 base pairs, 102, 104, 107, 110, 115, 120, 130, 140, 150, 170, 200, 230, 260, 400, 450, 500, 560, 620, or 690 base pairs up to 105, 165, 220, 275, 325, 375, 420, 465, 505, 540, 575, 605, 635, 660, 680, 690, or 700 base pairs.

Notably, the set of peaks is used to monitor quality, quantity, and fragmentation profile of the cfDNA extracted from the plasma, facilitating the assessment of integrity and suitability of the cfDNA for downstream analysis. Herein, the set of peaks representing the cfDNA fragments in the corrected cfDNA fragment profile is examined, specifically focusing on fragments ranging from 100 base pairs to 700 base pairs. The peak pattern and intensity are analysed to determine whether the cfDNA fragment profile matches the expected distribution, ensuring that no errors occurred during the extraction process (such as degradation or incomplete recovery of the cfDNA). The presence of the internal spike in the corrected cfDNA fragment profile further supports the accuracy of the results. If the set of peaks align with expected values based on known standards or reference samples, it indicates that the cfDNA is correctly quantified and fragmented. It will be appreciated that using the corrected cfDNA fragment profile to monitor the size distribution and concentration of the cfDNA fragments enhances the precision of the quantification process, ensuring reliable results for subsequent analysis. It will also be appreciated that focusing on the set of peaks within defined range of 100-700 base pairs allows for a more detailed and accurate assessment of the cfDNA, enabling the identification of potential errors or inconsistencies in the extraction process.

Throughout the present disclosure, the term “cell-free DNA concentration” refers to an amount of DNA present in a biological sample that circulates freely in an extracellular space, within the bloodstream, without being contained within cells. The cfDNA concentration, often released from dying or apoptotic cells, is a mixture of short DNA fragments that can be measured to assess the health status of the Canis familiaris. Herein, the concentration of the cfDNA in plasma from the Canis familiaris is measured to quantify the amount of the cfDNA present, which can provide valuable insights into the health status of the Canis familiaris. This measurement is typically performed using techniques for example, such as spectrophotometry, fluorometry, and similar. Notably, an accurate measurement of the cfDNA concentration is essential for ensuring reliable downstream analyses and for assessing the suitability of the cfDNA for diagnostic or research applications. It will be appreciated that accurately measuring the cfDNA concentration enables precise quantification of circulating DNA, which is essential for evaluating the health status of the Canis familiaris. It will also be appreciated that this measurement facilitates the assessment of quality of the cfDNA, ensuring its suitability for diagnostic or research purposes, for example, such as detection of diseases, monitoring of treatment responses, and evaluating biological conditions in the Canis familiaris.

Optionally, the cfDNA concentration below 10 pg/μl indicates a healthy subject. In this regard, the term “healthy subject” refers to the Canis familiaris that exhibits no clinical signs of disease or pathological conditions, with physiological markers, including the cfDNA concentration falling within the established normal ranges for the Canis familiaris. It will be appreciated that the establishment of the cfDNA concentration below 10 pg/μl as an indicator of the healthy subject enhances the specificity and sensitivity of diagnostic assays by providing a clear reference point for distinguishing between healthy and potentially diseased animals. It will also be appreciated that this threshold allows for early detection of abnormalities, enabling veterinarians to intervene at an earlier stage of disease progression, thereby improving treatment outcomes and overall animal health. Furthermore, it will be appreciated that the use of this threshold improves the reproducibility and accuracy of the cfDNA-based diagnostics, ensuring that the results are consistent across different laboratories and analytical platforms, leading to more reliable and standardized testing for the Canis familiaris. A technical effect of the aforementioned feature is that it enables a non-invasive and quantitative means to distinguish healthy Canis familiaris subjects from those potentially exhibiting pathological conditions, thereby enhancing the accuracy and reliability of diagnostic assessments based on the cfDNA concentration.

Optionally, the cfDNA concentration in the range of 10 pg/μl to 20 pg/μl and the corrected cell-free DNA fragment profile are used to indicate the presence of at least one of the following: cancer, chronic diseases, infections, inflammation, autoimmune diseases, organ damage. In this regard, the cancer in the Canis familiaris refers to a class of diseases characterized by the uncontrolled growth and division of abnormal cells that can invade surrounding tissues and, in some cases, spread (metastasize) to other parts of the body. Examples of different types of the cancer in the Canis familiaris may include, but are not limited to, lymphoma, osteosarcoma, mammary gland tumours, adenocarcinoma, hemangiosarcoma, histiocytic sarcoma, mast cell tumour, melanoma, and testicular cancer. The aforesaid types of the cancer in the Canis familiaris are well-known in the art. The infections in the Canis familiaris are conditions caused by pathogenic organisms for example, such as bacteria, viruses, fungi, parasites, and similar, that invade the body and disrupt normal biological functions. Examples of the infections in the Canis familiaris may include, but are not limited to, bacterial infections (like Staphylococcus), viral infections (like canine parvovirus), fungal infections (like blastomycosis), parasitic infections (like heartworm), and similar. The inflammation in the Canis familiaris refers to the body's immune response to harmful stimuli, such as pathogens, damaged cells, irritants, or similar. This response is marked by redness, swelling, heat, and pain in the affected area as the body attempts to eliminate harmful agents and begin the healing process. Typically, the inflammation can be either short-term and protective) or chronic (long-term and potentially damaging). Examples of the inflammation may include, but are not limited to, inflammatory conditions (like osteoarthritis), dermatitis (like skin inflammation), inflammatory bowel disease (IBD), and similar, where prolonged inflammation may lead to tissue damage and impact overall health of the Canis familiaris.

Herein, the cfDNA concentration range and the corrected cfDNA fragment profile in the blood sample are essential indicators of physiological and pathological conditions in the Canis familiaris. Levels of the cfDNA that are elevated above what is considered normal for healthy subjects often suggest cellular turnover and death, which are hallmarks of various pathological states. Thereby, analysing the cfDNA helps in identifying conditions like cancer, infections, inflammation, and similar, which all exhibit distinct changes in patterns of the cfDNA. Using these indicators helps the veterinarians and researchers to non-invasively assess health status of the Canis familiaris and detect diseases at an early stage. Herein, the cfDNA concentration range may, for example, lie in a range from 10, 12, 14, 16, or 19 pg/μl up to 11, 13, 15, 17, or 20 pg/μl. It will be appreciated that using the cfDNA concentration in the range of 10 pg/μl to 20 pg/μl, alongside the corrected cfDNA fragment profile, allows for a non-invasive, efficient method to indicate the presence of pathological conditions, improving diagnostic capabilities in the Canis familiaris. It will also be appreciated that this approach enables early detection of conditions such as cancer, infections, and inflammation, providing an advanced tool for veterinarians to monitor and intervene before the disease progresses to more severe stages. Furthermore, it will be appreciated that the combined analysis of the cfDNA concentration and the corrected cfDNA fragment profile minimizes false positives and false negatives, leading to more reliable diagnostics, which is essential in clinical and research settings. A technical effect of the aforementioned feature is that the method enables early and precise identification of the pathological conditions, such as the cancer, the infection, the inflammation, and similar, through quantitative and qualitative analysis of the cfDNA.

Optionally, the cfDNA concentration larger than 20 pg/μl indicates the presence of at least one of the following: cancer, infections, inflammation. In this regard, the cfDNA concentration, may for example, lie in a range from 20 pg/μl, 25, 30, 50, 70, 100 pg/μl, and so forth. Herein, when the cfDNA concentration is determined to exceed 20 pg/μl, it is flagged as a possible indicator of disease. Notably, the cfDNA concentration ranging from 10 pg/μl to 20 pg/μl may be considered outside breed-specific reference range. This concentration may be indicative of certain cancers, inflammatory conditions, and similar, where increased cfDNA release is associated with the heightened cellular turnover or immune response characteristic of these pathologies. The cfDNA concentration exceeding 20 pg/μl may be considered outside breed-specific reference range. While it is uncommon for inflammatory conditions to result in such high cfDNA levels, it may occur if the Canis familiaris is not undergoing treatment. However, the cfDNA concentration at or above the 20 pg/μl is more commonly associated with the cancer, as it reflects the heightened genomic instability and cellular turnover characteristic of malignancies. Thus, additional diagnostic tests or confirmatory tests may then be employed to identify specific conditions, leveraging this concentration threshold as a preliminary biomarker to guide further investigation. It will be appreciated that the cfDNA concentration above 10 pg/μl serves as an indicator of pathological conditions, enabling rapid, non-invasive screening in the Canis familiaris, thereby facilitating early intervention without the need for more intrusive diagnostic procedures. It will also be appreciated that this approach enables a more efficient allocation of veterinary resources, as elevated cfDNA levels can prompt timely and targeted follow-up testing, thereby improving the likelihood of identifying and treating diseases at the earlier stage. A technical effect of using the cfDNA concentration threshold above 10 pg/μl as a diagnostic marker is that it enables the early detection mechanism for pathological conditions in Canis familiaris without invasive procedures.

Optionally, the method further comprises measuring a gDNA contamination level. In this regard, the term “gDNA contamination level” refers to an amount of genomic DNA (gDNA) present within the cfDNA. The gDNA contamination level indicates the extent to which intact cellular DNA, which originates from lysed cells, is present alongside cfDNA, which circulates freely in extracellular spaces such as the blood plasma. Typically, the gDNA contamination level is quantified in terms of concentration (for example, pg/μl) or as a percentage relative to the total DNA content in the blood sample. The gDNA contamination level in the cfDNA can arise from a variety of sources, including insufficient separation of the plasma from blood cells, incomplete extraction procedures, or similar. When the gDNA contamination level is present, it can skew results, leading to false conclusions about the cfDNA concentration, the cfDNA fragment profile, and its association with health or disease in the Canis familiaris.

Herein, the gDNA contamination level is measured by quantifying the amount of gDNA present in the cfDNA to ensure the cfDNA is accurately represented without significant cellular DNA interference. This measurement can be performed using quantitative polymerase chain reaction (qPCR), nucleic acid quantification methods, and similar, where specific primers targeting known genomic DNA regions are used to differentiate the gDNA from the cfDNA. By comparing concentrations with standard reference samples, the gDNA contamination level may be assessed. This step helps to confirm the purity of the cfDNA, thereby supporting accurate downstream analyses and minimizing potential false signals due to cellular DNA fragments. It will be appreciated that measuring the gDNA contamination level allows for accurate quantification of the cfDNA by reducing the influence of cellular DNA, which is particularly essential in maintaining the integrity of cfDNA-based diagnostic results. It will also be appreciated that measuring the gDNA contamination level helps to validate the purity of the cfDNA, thereby improving the accuracy and reliability of downstream analyses (for example, such as cancer, infections, inflammation, and similar). A technical effect of measuring the gDNA contamination level is that it enhances the specificity and accuracy of analysis of the cfDNA by ensuring minimal interference from cellular DNA, thereby enabling more reliable diagnostics and insights into disease states in the Canis familiaris.

Optionally, the method further comprises comparing the monitored cell-free DNA fragment profile with a reference cell-free DNA fragment profile from a healthy subject. In this regard, the term “reference cell-free DNA fragment profile” refers to a characterized and quantified distribution of cfDNA fragment sizes and concentrations obtained from a biological sample, such as blood plasma, from the healthy subject. The reference cfDNA fragment profile serves as a baseline or standard for comparison in diagnostic analyses, allowing for the identification of deviations in the monitored cfDNA fragment profile that may indicate disease. Typically, the reference cfDNA fragment profile is established through consistent measurement methods to ensure reproducibility and reliability, capturing key fragment size peaks and overall fragment distribution specific to a healthy state in the Canis familiaris.

The method involves capturing the cfDNA fragment profile of the blood sample from the Canis familiaris under investigation and then performing a direct comparison with the cfDNA fragment profile of the healthy subject. Herein, the comparison may involve analysing several quantitative and qualitative features of the cfDNA fragment profile, such as fragment size distribution, peak intensity, and the pattern of peaks within specific base pair ranges. To perform this comparison, the cfDNA fragment profile from the healthy subject under examination is first generated, using high-resolution techniques, for example, such as capillary electrophoresis, next-generation sequencing (NGS), and similar.

This profile provides data on the abundance and distribution of the cfDNA fragments across a range of fragment sizes, usually between 100 to 700 base pairs. The reference cfDNA fragment profile is then obtained from the healthy subject of the Canis familiaris and serves as the baseline representation of expected cfDNA characteristics in a disease-free state.

The reference cfDNA fragment profile reflects typical cfDNA fragmentation patterns and concentrations expected in the healthy subject, encompassing factors such as common peak sizes and variability limits. During the comparison process, statistical methods may be employed to quantify deviations between the monitored cfDNA fragment profile and the reference cfDNA fragment profile. Increased variability in the monitored cfDNA profile for example, such as atypical fragment sizes, shifts in peak intensities, presence of new peaks not observed in the reference cfDNA profile, and similar, may signal disruptions in the cfDNA that are associated with pathological processes. Significant deviations from the reference cfDNA profile may indicate abnormal cellular turnover, genomic instability, or other underlying conditions, including oncogenic activity. It will be appreciated that comparing the monitored cfDNA fragment profile with the reference profile from the healthy subject enables the identification of deviations indicative of potential pathological conditions, such as the cancer, thereby facilitating early detection. It will also be appreciated that this approach provides a highly sensitive and non-invasive diagnostic tool, allowing for real-time assessment of genomic instability and abnormal cellular turnover without the need for more invasive biopsy procedures.

Optionally, the method further comprises storing the blood sample at a temperature range from 2° C. up to 8° C. before separating the plasma, and the separation is carried out over a period of at least 240 minutes. In this regard, the temperature may, for example, lie in a range from 2, 3, 4, 5, 6, or 7° C. up to 3, 5, 6, 7, or 8° C. After the blood sample is collected from the Canis familiaris, it is immediately stored in a cooling environment, maintaining the temperature between 2° C. and 8° C. This temperature range helps to preserve the cfDNA by preventing premature cellular lysis and reducing the activity of nucleases that could degrade the cfDNA. The blood sample is then processed by separating the plasma from the blood cells over a period of at least 240 minutes. Specifically, time range of separating the plasma from the blood sample may, for example, lie in a range from 1 minute, 2, 3, 5, 7, 10, 15, 20, 30, 40, 60, 80, 110, 140, 170, 200, or 235 minutes up to 5, 55, 100, 140, 170, 195, 215, 225, 230, 234, 236, 237, 238, or 240 minutes. The plasma is separated through standard centrifugation protocols to isolate the cfDNA for further analysis, ensuring minimal degradation during this critical period. By adhering to this time frame, the method preserves the integrity of the cfDNA and ensures that it remains representative of the true biological state of the Canis familiaris. Most importantly, this time frame allows to reduce contamination with genomic DNA (gDNA) from white blood cells that will influence the results of the method. It will be appreciated that storing the blood sample at a controlled temperature range of 2° C. to 8° C. before plasma separation ensures the preservation of integrity of the cfDNA, thereby minimizing degradation or contamination that could compromise diagnostic accuracy. It will also be appreciated that maintaining this temperature range allows for more reliable and consistent analysis of the cfDNA, leading to improved detection of potential health conditions in the Canis familiaris. Furthermore, over a period of at least 240 minutes time frame for the separation of the plasma offers an optimal balance between sample stability and practical processing, thereby enhancing overall efficiency and reliability of the method. A technical effect of storing the blood sample within the range of 2° C. to 8° C. and separating the plasma from the blood sample over a period of at least 240 minutes, ensures the preservation of cfDNA integrity, which improves the accuracy and consistency of downstream analyses.

### EXPERIMENTAL PART

In an exemplary experimental part, an effect of breed and health status on the cfDNA concentration and cfDNA fragment profile in the Canis familiaris was assessed. Blood samples were collected by veterinary professionals with informed consent from each dog's owner. Information was collected from owners, wherein the information comprised at least one of: an age, a breed, a sex, a spay status or neuter status, any known clinical condition, any suspected clinical condition. Over 2,000 healthy control dogs, 600 dogs with non-cancer diseases, and 400 cancer cases across five European countries were tested demonstrating its applicability across multiple clinical scenarios. Herein, the multiple clinical scenarios comprise at least one of: disease screening, diagnostic support, targeted treatment selection, detection of minimal residual disease (MRD), treatment monitoring, recurrence tracking. Healthy dogs aged two years or older and not pregnant. Dogs with non-cancer conditions (like osteoarthritis, heart disease, kidney disease, thyroid disease, epilepsy, and similar) were also included to evaluate test performance in the presence of non-cancer-related clinical conditions. There were no restrictions regarding age or breed but most dogs were at least 2 years old.

For each dog, a minimum of 3 milliliter (ml) of whole blood (yielding greater than 1 ml plasma) was collected from one of the following: the vena cephalis, the vena saphena lateralis, the vena jugularis. The blood samples were stored in Roche circulating cfDNA tubes or EDTA tubes. The blood samples were at the temperature range from 2° C. up to 8° C. before separating the plasma, and the separation of the plasma was carried out within at least 240 minutes. Then, the plasma was transferred to clean DNA LoBind Eppendorf tubes and stored frozen before being transported. The blood samples were then shipped across different regions to the research laboratory. Upon arrival, said samples were assessed for hemolysis, lipemia, and other quality-affecting features, and information was logged in an internal database. Samples were then stored frozen until extraction of the cfDNA. Prior to processing, the blood samples were centrifuged to remove any remaining cell debris and were aliquoted into 1 ml portions. One aliquot was used for the cfDNA extraction. Notably, data of the plasma is logged into a data portal to track information, wherein the data portal refers to an online or software-based platform where the data of the plasma and associated information are stored, managed, and tracked throughout the process.

In this regard, the Qiagen QiaAmp® circulating cell-free DNA mini kit was employed for most extractions due to its reliability with small input volumes such as 500 microlitres (μl) to 2 millilitres (ml). Before extraction, each plasma sample was spiked with the internal spike of known size and concentration to correct for variations in extraction efficiency across samples and batches. Then, the extracted cfDNA was diluted to 50 μl in the elution buffer. For quality control, 2 μl was analysed for the cfDNA concentration, the gDNA contamination, and fragment size distribution using Agilent Tapestation. Thereby, data from the cfDNA concentrations for healthy dogs were compiled to establish breed-specific reference ranges for each age group, facilitating the assessment of normal cfDNA variability within each breed. These breed-specific profiles were incorporated into the test's diagnostic criteria, allowing for adjustments based on breed and age. Once a protocol had been established that generated reliable and reproducible cfDNA concentration values, the test was run on samples collected from dogs that were clinically healthy and dogs with verified clinical diagnosis. Of the dogs with verified clinical diagnoses, 75-100% of samples were outside the reference range, depending on the clinical condition. It should be noted that the actual cfDNA concentration values have a wide dynamic range. TABLE 1 below summarises the mean cfDNA concentrations for a set of clinical conditions. Compared to samples from clinically healthy dogs, inflammatory and chronic diseases contained on average twice as much cfDNA; osteoarthritis contained on average four times as much cfDNA; Benign tumours, mast cell tumours, and melanomas contained on average twice as much cfDNA; Mammary carcinomas, osteosarcomas, soft tissue sarcomas, and testicular cancers contained on average three times as much cfDNA and hemangiosarcomas, histiocytic sarcomas, lymphomas and renal cancers contained on average at least 10 times as much cfDNA.

TABLE 1

| Diagnosis | Mean cfDNA (ng/ml) |
|---|---|
| Hemangiosarcoma | 48 |
| Histiocytic sarcoma | 356 |
| Lymphoma | 371 |
| Mammary carcinoma | 15 |
| Mast cell tumor | 10 |
| Melanoma | 11 |
| Osteosarcoma | 21 |
| Renal cancer | 62 |
| Soft tissue sarcoma | 18 |
| Testicular cancer | 13 |
| Benign tumor | 11 |
| Epilepsy | 12 |
| Heart disease | 11 |
| IBD | 8 |
| Kidney disease | 11 |
| Osteoarthritis | 20 |

Variations in cfDNA concentration within a specific clinical condition correlated with the disease severity, such as the disease progression (e.g. staging in cancer) and the size of the affected area (e.g. the number of affected limbs in osteoarthritis).

Measuring cfDNA can be used for monitoring disease treatment. cfDNA concentration of the first sample was used as the baseline, and all subsequent samples were compared to the previous samples to form a trend. The oncologists treating dogs used these trends to decide whether to continue, discontinue, or change the treatment regime. Some examples of these different trends have been collected in Table 2. The first example is of a dog that died soon after the fifth sample was taken, for the others, only three samples have been taken.

TABLE 2

| | T1 | T2 | T3 | T4 | T5 |
|---|---|---|---|---|---|
| Dog | (ng/ml) | (ng/ml) | (ng/ul) | (ng/ul) | (ng/ml) |
| 5 Y female Golden | | 27 | 15 | 65 | 215 |
| Retriever, Renal | | | | | |
| cancer | 45 | | | | |
| 16 Y female Basenji, | 4 | 10 | 19 | tbd | tbd |
| Mammary cancer | | | | | |
| 11 Y female Belgian | 15 | 14 | 9 | tbd | tbd |
| Groenendael, | | | | | |
| Mammary cancer | | | | | |
| 6 Y female Labrador | 21 | 20 | 20 | tbd | tbd |
| Retriever, | | | | | |
| Lymphoma | | | | | |
| 11 Y female Cocker | 40 | 5 | 10 | tbd | tbd |
| Spaniel, Lymphoma | | | | | |
| 10 Y male Flat- | | | | | |
| coated Retriever, | | | | | |
| Histiocytic sarcoma | 46 | 12 | 6 | tbd | tbd |

Multiple trends were seen: In the first group, there was a rapid decrease of cfDNA concentration after the treatment started, and the level continued decreasing throughout the treatment. In the second group, cfDNA concentration decreased in the beginning of the treatment but started increasing again. Some dogs in this group unfortunately died soon after but others started to receive another treatment combination with a better response. In the third group, cfDNA concentration stayed stable during the treatment.

Moreover, essential confounding factors affecting performance of the test were severe haemolysis and lacking diagnosis information to interpret results correctly. Additional factors, such as ongoing infections (like pneumonia), severe inflammation (like osteoarthritis), recent surgical procedures, severe lipemia, and undisclosed cancer treatments (like chemotherapy, radiotherapy, or palliative care), also influenced test outcomes. The most critical factors that affected the test performance were the type of sample collection tube used, the cfDNA extraction method type, and variation in quality control (QC) steps until optimal protocols for all steps were developed.

Few example cases that were identified, a sample was received from a presumably healthy dog. The test initially showed a high cfDNA concentration, which raised concerns about advanced lymphoma. However, further analysis revealed that the dog had recently undergone tail amputation due to injury. In this regard, a follow-up test was conducted one month later which showed a 100-fold reduction in the cfDNA concentration level, though levels still remained slightly above the cancer threshold. Nearly a year later, the dog was euthanized due to a large soft tissue sarcoma, confirmed by histopathology. This case, along with 26 similar cases of elevated cfDNA concentration in healthy dogs, led to cancer diagnoses (for example, such as hemangiosarcoma, histiocytic sarcoma, osteosarcoma, lymphoma, and other cancer types) within 4 to 6 months. These findings highlight the importance of taking multiple samples over time and the need for continuous monitoring, as various factors, such as recent surgeries or injuries, can influence the cfDNA levels. Monitoring the cfDNA overtime allows for more accurate interpretation of results, distinguishing between transient increases due to non-cancerous events and sustained elevations that may indicate the onset of disease.

Secondly, two samples were received from dogs diagnosed with lymphoma by two different clinics. However, the test results showed no signs of the cancer, leading to a report indicating that the cancer signal was not detected. In this regard, a month later, both clinics confirmed that the dogs did not have lymphoma, and the initial diagnoses were found to be false alarms. This highlights an accuracy of the test in comparison to traditional diagnostic methods, demonstrating its ability to avoid false positives and provide reliable results where previous methods may lead to misdiagnosis.

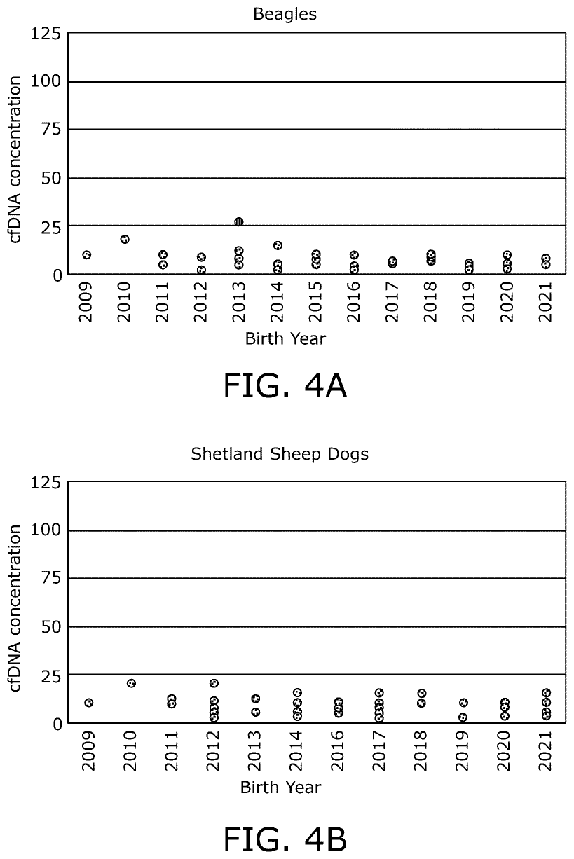

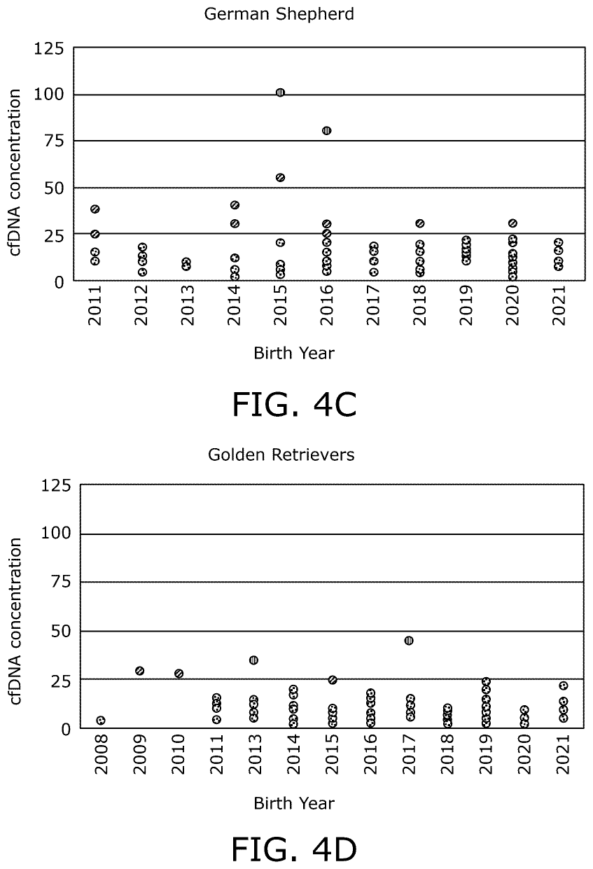

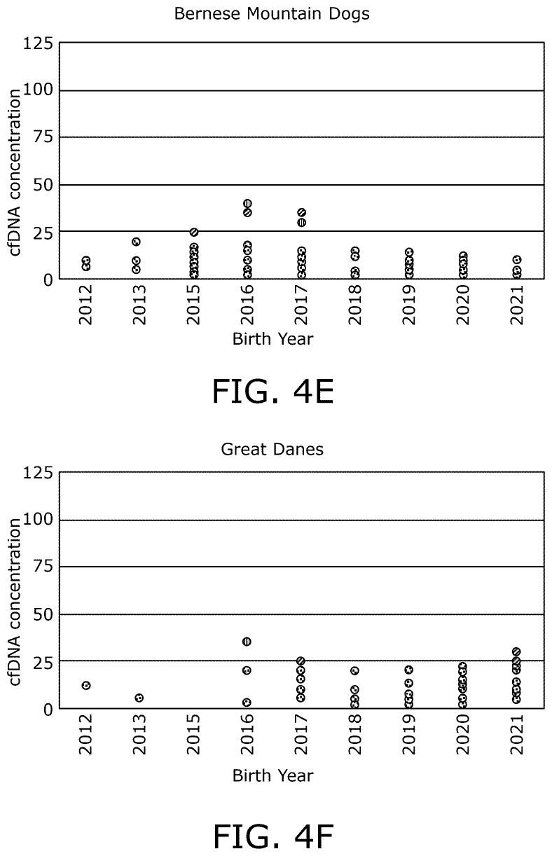

Moreover, prior testing methods have generally applied a single cutoff threshold for the cfDNA concentration, assuming uniform cfDNA levels across different dog breeds and cancer types. Notably, the single cutoff threshold refers to a fixed concentration level of the cfDNA that is used as a threshold to distinguish between normal and abnormal conditions. However, it was observed that the cfDNA levels vary by breed, influencing determination of the normal cfDNA range for each breed. To address this variability, breed-specific profiles were developed for breeds from which at least 50 dogs with high-quality samples were sourced, ensuring balanced representation across sexes and age groups (as explained in conjunction with FIGS. 4A, 4B, 4C, 4D, and 4E).

In conclusion, the test for measuring the cfDNA concentration in the Canis familiaris has demonstrated strong reliability and clinical utility in the detection, diagnosis, and monitoring of a wide range of the canine cancers and other diseases. The breed-specific approach of the test accounts for natural cfDNA variation among breeds, enhancing diagnostic accuracy and establishing relevant cutoff values. Evaluation across multiple animal hospitals in Slovenia, France, Netherland, Poland, and Finland has confirmed effectiveness of the test in both early detection and recurrence monitoring.

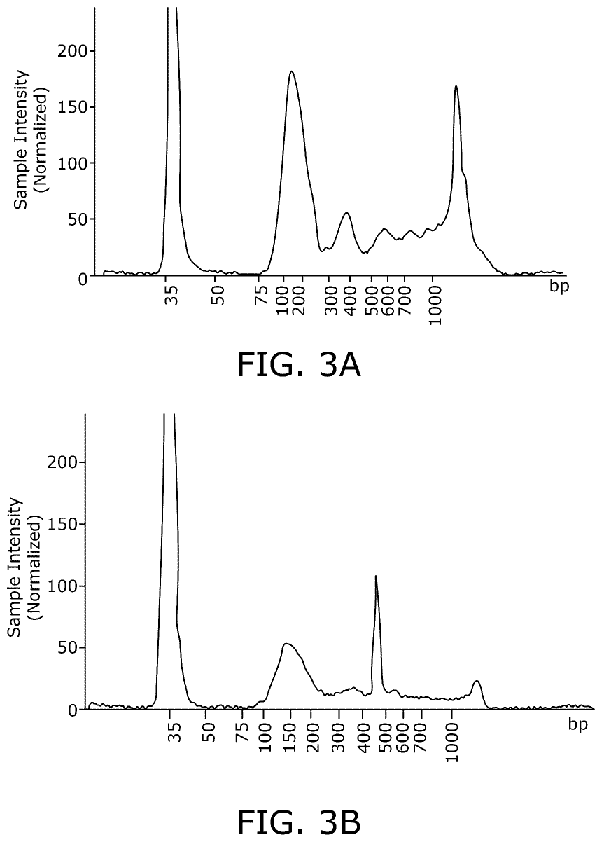

The experimental part can be studied in conjunction with FIGS. 3A, 3B, 3C, 3D, 3E, 3F, 4A, 4B, 4C, 4D, 4E, and

### DETAILED DESCRIPTION OF THE DRAWINGS

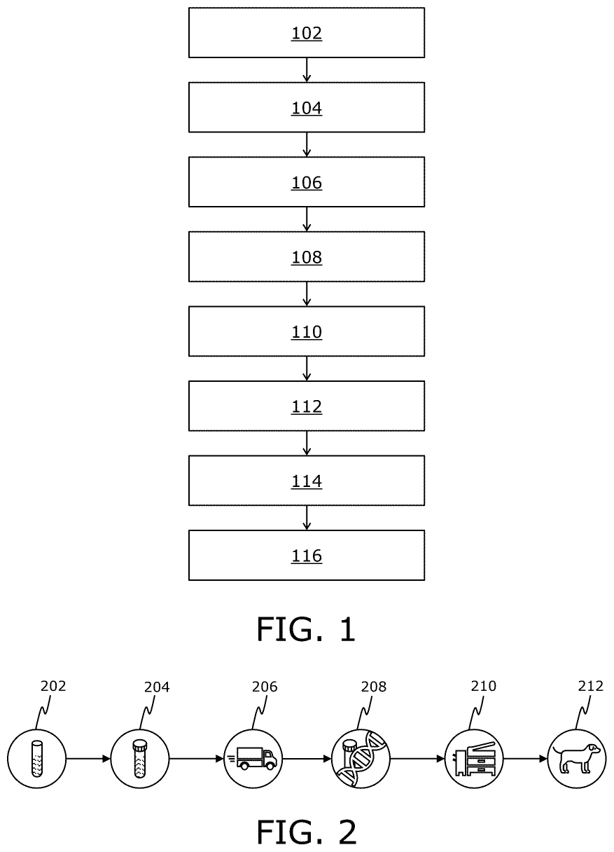

Referring to FIG. 1, illustrated are steps of a method of measuring a cell-free DNA in a blood of Canis familiaris, in accordance with an embodiment of the present disclosure. At step 102, a plasma from a blood sample of Canis familiaris is separated. At step 104, the separated plasma is spiked with an internal spike. At step 106, a cell-free DNA from the spiked plasma is extracted. At step 108, a cell-free DNA fragment profile of the cell-free DNA is measured. At step 110, the internal spike in the cell-free DNA fragment profile is confirmed. At step 112, the cell-free DNA fragment profile with the internal spike is corrected. At step 114, the corrected cell-free DNA fragment profile is monitored, wherein the cell-free DNA fragment profile comprises a set of peaks that range from 100 base pairs up to 700 base pairs. At step 116, a cell-free DNA concentration is measured.

The aforementioned steps are only illustrative and other alternatives can also be provided where one or more steps are added, one or more steps are removed, or one or more steps are provided in a different sequence without departing from the scope of the claims herein.

Referring to FIG. 2, illustrated is an exemplary workflow of processing steps for monitoring a cell-free DNA in a blood sample of Canis familiaris, in accordance with an embodiment of the present disclosure. With reference to FIG. 2, at step 202, the blood sample is taken from at least one Canis familiaris (depicted as Canis familiaris) and is processed to separate a plasma. At step 204, the plasma from the blood sample of the Canis familiaris is separated. Herein, the plasma sample is stored at −20° C. until it is ready for shipment. At step 206, the plasma sample is batched for shipping via courier. Notably, the plasma sample is transported in batches. At step 208, a diagnostic center receives the plasma sample and determines next steps for the Canis familiaris. At step 210, a cell-free DNA is extracted from the plasma sample, and quality control checks are performed. The extracted cell-free DNA is subjected to a cancer detection test. At step 212, test results are analyzed, incorporating the Canis familiaris and breed-specific data to enhance diagnostic accuracy. The final results are transmitted back to a clinic for review, supporting informed decision-making for the Canis familiaris.

FIG. 2 is merely an example, which should not unduly limit the scope of the claims herein. A person skilled in the art will recognize many variations, alternatives, and modifications of embodiments of the present disclosure.

Referring to FIGS. 3A, 3B, 3C, 3D, 3E, and 3F, illustrated is a graphical representation of cancer type-specific cell-free DNA (cfDNA) fragment size distributions for various canine cancers, in accordance with an embodiment of the present disclosure. Specifically, FIG. 3A visualizes the cfDNA fragment size profile for lymphoma cancer type, FIG. 3B visualizes the cfDNA fragment size profile for histiocytic sarcoma cancer type, FIG. 3C visualizes the cfDNA fragment size profile for osteosarcoma cancer type, FIG. 3D visualizes the cfDNA fragment size profile for hemangiosarcoma cancer type, FIG. 3E visualizes the cfDNA fragment size profile for mast cell tumor cancer type, and FIG. 3F displays the cfDNA fragment size distribution for melanoma cancer type. The cfDNA fragment size profile in the FIGS. 3A-3F highlight the cfDNA fragment sizes and peak concentrations indicative of aforementioned cancer types. Herein, a vertical axis (depicted as Y-axis) represents DNA fragment peak concentration (which is sample intensity as shown), and a horizontal axis (depicted as X-axis) represents DNA fragment sizes in base pairs (bp).

In relation to the cfDNA fragment profiles shown in FIGS. 3A and 3B, distinct cfDNA patterns are observable across different cancer types, with notable variations in the cfDNA concentration and fragment sizes for certain cancers. These cancer types in FIGS. 3A and 3B showed high to very high peaks at 150-200 bp and lower peaks around 300 bp and 450 bp, giving the cfDNA fragment profile appearance of a rollercoaster. Notably, the term rollercoaster refers to the cfDNA fragment profile with multiple peaks and multiple valleys, resembling ups and downs of rollercoaster track.

In FIG. 3A, the lymphoma cancer type shows a pronounced peak observed within the 150-200 bp range, followed by secondary peaks around 300 bp and 450 bp, creating a rollercoaster-like pattern. The rollercoaster indicates that the cfDNA concentration fluctuates noticeably across different fragment sizes rather than following a smooth or uniform distribution. In FIG. 3B, the melanoma cancer type exhibits a comparable profile, with high intensity in the 150-200 bp range, accompanied by moderate peaks at 300 bp and 450 bp, thus contributing to overall rollercoaster-like cfDNA fragment profile.

Overall, these cfDNA fragment profiles reveal variability that may reflect underlying tumor characteristics, for example, such as size, location, growth rate, and similar.

FIGS. 3A and 3B are merely examples, which should not unduly limit the scope of the claims herein. A person skilled in the art will recognize many variations, alternatives, and modifications of embodiments of the present disclosure.

Referring to FIGS. 4A, 4B, 4C, 4D, 4E, and 4F illustrated is a graphical representation of breed-specific cell-free DNA concentration levels in healthy controls across different breeds of Canis familiaris, in accordance with an embodiment of the present disclosure. As shown, samples of Canis familiaris that are presumably healthy are shown circles with dotted hatching and diagonal striped hatching, whereas circles with horizontal hatching represent samples from Canis familiaris diagnosed with cancer. Notably, circles with diagonal strip hatching represent presumably healthy dogs with the cfDNA concentration level being too similar to diagnosed cancer cases. These breed-specific profiles include examples for small, medium, and large dog breeds, highlighting a distinct cell-free DNA range for each breed with sufficient healthy control samples. Herein, a vertical axis (depicted as Y-axis) represents cell-free DNA concentration levels, and a horizontal axis (depicted as X-axis) represents birth year of each Canis familiaris.

Specifically, FIG. 4A shows the cell-free DNA concentration level for Beagles. In the birth year 2013 for the Beagles, there is shown a circle with horizontal hatching representing diagnosed cancer sample with the cfDNA concentration level of 25 picograms per microliter (pg/μL). This cancer-affected sample demonstrates an elevated cfDNA level, which can be visually compared to the cfDNA range in the healthy control samples. The visual comparison between the diagnosed cancer sample and the surrounding healthy samples emphasizes the increased cfDNA concentration seen in the cancer-affected Beagle, highlighting a potential indicator for the cancer detection in this breed.

FIG. 4B shows the cell-free DNA concentration level for Shetland Sheep Dogs. This figure includes only circles with dotted hatching, which represent presumably healthy dogs. Notably, circles with large dotted hatching are present in this figure, indicating that all the samples for the Shetland Sheep Dogs fall within the healthy control range, with no samples diagnosed with cancer. This suggests a stable and consistent cfDNA concentration level across the healthy Shetland Sheep Dog population represented in data.

FIG. 4C shows the cell-free DNA concentration level for German Shepherds. There are observed both the presumably healthy German Shepherds and diagnosed with cancer. The presence of both healthy controls and cancer-affected samples highlights variability in the cfDNA concentrations across the German Shepherd population. This figure shows both presumably healthy dogs and those diagnosed with cancer.

The data reveals variability in the cfDNA concentration across the German Shepherd population, with healthy controls exhibiting the cfDNA levels primarily between 0 pg/μL and 20 pg/μL. Cancer-affected samples display elevated the cfDNA concentration, are observed at 75 pg/μl and 100 pg/μL, indicating significantly higher levels typically associated with cancer. The presence of both the healthy and cancer-diagnosed samples highlights the variation in the cfDNA concentrations within this breed.

FIG. 4D shows the cell-free DNA concentration level for Golden Retrievers. There are observed both the presumably healthy Golden Retrievers and diagnosed with cancer. The presence of both healthy controls and cancer-affected samples highlights variability in the cfDNA concentrations across the Golden Retrievers population. This figure shows both presumably healthy dogs and those diagnosed with cancer. The data reveals variability in the cfDNA concentration across the Golden Retrievers population, with healthy controls exhibiting cfDNA levels primarily between 0 pg/μL and 20 pg/μL. Cancer-affected samples display elevated the cfDNA concentration, observed at 30 pg/μL and 40 pg/μL, indicating significantly higher levels typically associated with cancer. The presence of both the healthy and cancer-diagnosed samples highlights the variation in the cfDNA concentrations within this breed.

FIG. 4E shows the cell-free DNA concentration level for Bernese Mountain Dogs. There are observed both the presumably healthy Bernese Mountain and diagnosed with cancer. The presence of both healthy controls and cancer-affected samples highlights variability in the cfDNA concentrations across the Bernese Mountain population. This figure shows both presumably healthy dogs and those diagnosed with cancer. The data reveals variability in the cfDNA concentration across the Bernese Mountain population, with healthy controls exhibiting cfDNA levels primarily between 0 pg/μL and 20 μg/L. Cancer-affected samples display elevated the cfDNA concentration, and are observed at 30 pg/μL and 40 pg/μL, indicating significantly higher levels typically associated with cancer. The presence of both the healthy and cancer-diagnosed samples highlights the variation in the cfDNA concentrations within this breed.

FIG. 4F shows the cell-free DNA concentration level for Great Danes. There are observed both the presumably healthy Great Danes and diagnosed with cancer. The presence of both healthy controls and cancer-affected samples highlights variability in the cfDNA concentrations across the Great Danes population. This figure shows both presumably healthy dogs and those diagnosed with cancer. The data reveals variability in the cfDNA concentration across the Great Danes population, with healthy controls exhibiting cfDNA levels primarily between 0 pg/μL and 20 pg/μL. Cancer-affected samples display elevated the cfDNA concentration, are observed at 30 pg/μL, indicating significantly higher levels typically associated with the cancer. The presence of both the healthy and cancer-diagnosed samples highlights the variation in the cfDNA concentrations within this breed.

Overall, the analysis revealed that breed-specific cutoff thresholds are more effective in improving test accuracy compared to a single cutoff value. In breeds such as in the Beagles, where the cfDNA concentration in healthy controls were tightly clustered, setting a cutoff for detecting cancer is straightforward. However, for breeds with higher natural variability in the cfDNA levels, such as in the Bernese Mountain Dogs (BMDs) and Flat-Coated Retrievers (FCRs) (not explicitly shown but implied by breed variability), defining a reliable cutoff is more complex. These breeds show a wide range of healthy cfDNA concentrations, making it harder to distinguish cancer-affected dogs from healthy controls without accounting for breed-specific characteristics.

FIGS. 4A-F are merely examples, which should not unduly limit the scope of the claims herein. A person skilled in the art will recognize many variations, alternatives, and modifications of embodiments of the present disclosure.

1. A method of measuring a cell-free DNA in a blood of Canis familiaris, wherein the method comprises:
separating a plasma from a blood sample of Canis familiaris;
spiking the separated plasma with an internal spike;
extracting a cell-free DNA from the spiked plasma;
measuring a cell-free DNA fragment profile of the cell-free DNA;
confirming the internal spike in the cell-free DNA fragment profile;
correcting the cell-free DNA fragment profile with the internal spike;
monitoring the corrected cell-free DNA fragment profile, wherein the cell-free DNA fragment profile comprises a set of peaks that range from 100 base pairs up to 700 base pairs; and
measuring a cell-free DNA concentration.

2. The method according to claim 1, further comprising measuring a gDNA contamination level.

3. The method according to claim 1, wherein the cfDNA concentration below 10 pg/μl indicates a healthy subject.

4. The method according to claim 1, wherein the cfDNA concentration in the range of 10 pg/μl to 20 pg/μl and the corrected cell-free DNA fragment profile are used to indicate the presence of at least one of the following: cancer, chronic disease, infection, inflammation, autoimmune disease, organ damage.

5. The method according to claim 1, wherein the cfDNA concentration larger than 20 pg/μl indicates the presence of at least one of the following: cancer, chronic disease, infections, inflammation, autoimmune disease, organ damage.

6. The method according to claim 1, wherein the blood sample is collected from one of the following: vena cephalis, vena saphena lateralis, vena jugularis.

7. The method according to claim 1, further comprising storing the blood sample at a temperature range from 2° C. up to 8° C. before separating the plasma, and the separation is carried out over a period of at least 240 minutes.

8. The method according to claim 1, wherein the extracted cell-free DNA is diluted with an elution buffer.
